(19) United States
(12) Patent Application Publication
(10) Pub. No.: US 2018/0168154 A1
(43) Pub. Date: Jun. 21, 2018

(54) PLANT GROWTH REGULATING COMPOUNDS

(71) Applicant: Syngenta Participations AG, Basel (CH)

(72) Inventors: Alain DE MESMAEKER, Stein (CH); Mathilde Denise LACHIA, Stein (CH); Alexandre Franco Jean Camille LUMBROSO, Stein (CH); Stefano RENDINE, Stein (CH); Claudio SCREPANTI, Stein (CH)

(73) Assignee: Syngenta Participations AG, Basel (CH)

(21) Appl. No.: 15/578,941

(22) PCT Filed: Jun. 1, 2016
(86) PCT No.: PCT/EP2016/062348

§ 371 Date: Dec. 1, 2017

(30) Foreign Application Priority Data
Jun. 3, 2015 (EP) 1509624.1

(51) Int. Cl. A01N 43/38; C07D 405/12

(52) U.S. Cl. CPC A01N 43/38; C07D 405/12

(57) ABSTRACT

Compounds of the Formula (I) wherein the substituents are as defined in claim 1, useful as plant growth regulators, particular in seed germination.

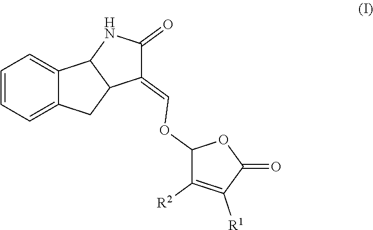

The present invention relates to novel strigolactam derivatives, to processes for preparing these derivatives including intermediate compounds, to plant growth regulator or seed germination promoting compositions comprising these derivatives and to methods of using these derivatives in controlling the growth of plants and/or promoting the germination of seeds.

Strigolactone derivatives are phytohormones which may have plant growth regulation and seed germination properties. They have previously been described in the literature. Certain known strigolactam derivatives (eg, see WO 2012/080115), may have properties analogous to strigolactones, eg, plant growth regulation and/or seed germination promotion.

For such compounds to be used, in particular, in seed treatment applications (eg, as seed coating components), hydrolytic stability and soil stability are important once a seed has been planted in the field in terms of maintaining the compound's biological activity.

According to the present invention, there is provided a compound of Formula (I):

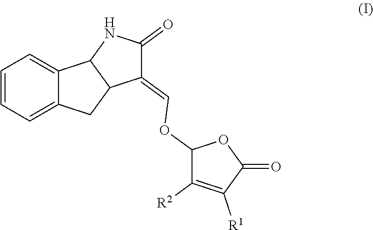

wherein
R¹ is C₁-C₃alkyl; and
R² is C₁-C₃alkyl or C₁-C₃alkoxy;
or salts or N-oxides thereof.

The compounds of Formula (I) have been shown to possess superior hydrolytic stability and soil stability compared to known strigolactam derivatives, while retaining seed germination properties.

In a second aspect of the invention, there is provided a plant growth regulator or seed germination promoting composition, comprising the compound according to the present invention, and optionally, an agriculturally acceptable formulation adjuvant.

In a third aspect of the invention, there is provided a method for regulating the growth of plants at a locus, wherein the method comprises applying to the locus a plant growth regulating amount of the composition according to the second aspect of the invention.

In a fourth aspect of the invention, there is provided a method for promoting the germination of seeds comprising applying to the seeds, or a locus containing seeds, a seed germination promoting amount of a composition according to the second aspect of the invention.

In a fifth aspect of the invention, there is provided a method for controlling weeds, comprising applying to a locus containing weed seeds, a seed germination promoting amount of a composition according to the second aspect of the invention, allowing the seeds to germinate, and then applying to the locus a post-emergence herbicide.

In a sixth aspect of the invention, there is provided the use of a compound of Formula (I) according to the invention as a plant growth regulator or a seed germination promoter.

In a seventh aspect of the invention, there is provided a method of treating a plant propagation material comprising applying to the plant propagation material a composition according to the invention in an amount effective to promote germination and/or regulate plant growth.

In an eighth aspect of the invention, there is provided a plant propagation material treated with a compound of Formula (I) according to the invention, or a composition according to the invention.

In a ninth aspect of the invention, there is provided a compound according to Formula (II):

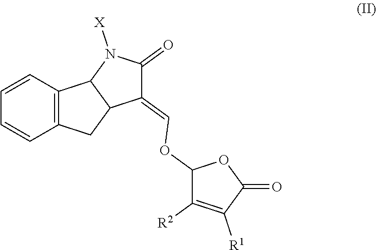

wherein

X is a protecting group;

R¹ is C₁-C₃alkyl; and

R² is C₁-C₃alkyl or C₁-C₃alkoxy.

The present invention may also provide a method for improving the tolerance of a plant to abiotic stress factors. ‘Abiotic stress factors’ are factors which cause sub-optimal growing conditions such as drought (e.g. any stress which leads to a lack of water content in plants, a lack of water uptake potential or a reduction in the water supply to plants), cold exposure, heat exposure, osmotic stress, UV stress, flooding, increased salinity (e.g. in the soil), increased mineral exposure, ozone exposure, high light exposure and/or limited availability of nutrients (e.g. nitrogen and/or phosphorus nutrients). A plant with improved tolerance to stress factors may have an increase in any of the aforementioned traits or any combination or two or more of the aforementioned traits. In the case of drought and nutrient stress, such improved tolerances may be due to, for example, more efficient uptake, use or retention of water and nutrients. In particular, the compounds or compositions of the present invention are useful to improve plant (eg, maize) tolerance to cold stress, eg, at temperatures from 5 to 15° C. Compounds of Formula (I) may be used under drought stress conditions or cold stress conditions for corn seed germination.

The compounds of Formulae (I) and (II) may exist in different geometric or optical isomers (diastereoisomers and enantiomers) or tautomeric forms. This invention covers all such isomers and tautomers and mixtures thereof, in all proportions, as well as isotopic forms, such as deuterated compounds. The invention also covers all salts, N-oxides, and metalloidic complexes of the compounds of Formula (I) and (II).

As used herein, the term “C₁-C₆ alkyl” refers to a straight or branched hydrocarbon chain radical consisting solely of carbon and hydrogen atoms, containing no unsaturation, having from one to six carbon atoms, and which is attached to the rest of the molecule by a single bond. The term “C₁-C₃ alkyl” is to be construed accordingly. Examples of C₁-C₆ alkyl include, but are not limited to, methyl, ethyl, n-propyl, 1-methylethyl (isopropyl), n-butyl, 1-dimethylethyl (tert-butyl) and n-pentyl.

As used herein, the term “C₂-C₆ alkenyl” refers to a straight or branched hydrocarbon chain radical group consisting solely of carbon and hydrogen atoms, containing at least one double bond that can be of either the (E)- or (Z)-configuration, having from two to six carbon atoms, which is attached to the rest of the molecule by a single bond. Examples of C₂-C₆ alkenyl include, but are not limited to, ethenyl, prop-1-enyl, but-1-enyl.

As used herein, the term “C₂-C₆ alkynyl” refers to a straight or branched hydrocarbon chain radical group consisting solely of carbon and hydrogen atoms, containing at least one triple bond, having from two to six carbon atoms, and which is attached to the rest of the molecule by a single bond. Examples of C₂-C₆ alkynyl include, but are not limited to, ethynyl, prop-1-ynyl, but-1-ynyl.

As used herein, the term “C₁-C₆ alkoxy” refers to a radical of the formula —ORₐ where Rₐ is a C₁-C₆ alkyl radical as generally defined above. The term “C₁-C₃ alkoxy” is to be construed accordingly. Examples of C₁-C₆ alkoxy include, but are not limited to, methoxy, ethoxy, propoxy, isopropoxy, butoxy.

As used herein, the term “C₁-C₆ haloalkyl” refers to a C₁-C₆ alkyl radical as generally defined above substituted by one or more of the same or different halogen atoms. Examples of C₁-C₆ haloalkyl include, but are not limited to fluoromethyl, 2-fluoroethyl, trifluoromethyl, 2,2,2-trifluoroethyl.

As used herein, cyano means a —CN group.

As used herein, hydroxy means an —OH group.

As used herein, amino means an —NH₂ group.

As used herein, the term “N—C₁-C₆ alkylamino” refers to a radical of the formula —NH—Rₐ where Rₐ is a C₁-C₆ alkyl radical as defined above.

As used herein, the term “N,N-di-C₁-C₆ alkylamino” refers to a radical of the formula —N(Rₐ)—Rₐ where each Rₐ is a C₁-C₆ alkyl radical, which may be the same or different, as defined above.

As used herein, the term “C₁-C₆ alkylcarbonyl” refers to a radical of the formula —C(═O)—Rₐ where Rₐ is a C₁-C₆ alkyl radical as defined above. Examples of C₁-C₆ alkylcarbonyl include, but are not limited to, acetyl.

As used herein, the term “C₁-C₆ alkoxycarbonyl” refers to a radical of the formula —C(═O)—O—Rₐ where Rₐ is a C₁-C₆ alkyl radical as defined above. The term “C₁-C₄ alkoxycarbonyl” is to be construed accordingly. Examples of C₁-C₆ alkoxycarbonyl include, but are not limited to, methoxycarbonyl, ethoxycarbonyl, isopropoxycarbonyl and tert-butoxycarbonyl.

As used herein, the term “aryl” refers to an aromatic ring system consisting solely of carbon and hydrogen atoms which may be mono-, bi- or tricyclic. Examples of such ring systems include phenyl, naphthalenyl, anthracenyl, indenyl or phenanthrenyl.

As used herein, the term “heteroaryl” refers to a 5- or 6-membered aromatic monocyclic ring radical which comprises 1, 2, 3 or 4 heteroatoms individually selected from nitrogen, oxygen and sulfur. The heteroaryl radical may be bonded via a carbon atom or heteroatom. Examples of heteroaryl include, but are not limited to, furyl, pyrrolyl, thienyl, pyrazolyl, imidazolyl, thiazolyl, isothiazolyl, oxazolyl, isoxazolyl, triazolyl, tetrazolyl, pyrazinyl, pyridazinyl, pyrimidyl or pyridyl.

As used herein, the term “heterocyclyl” or “heterocyclic” refers to a stable 5- or 6-membered non-aromatic monocyclic ring radical which comprises 1, 2, or 3, heteroatoms individually selected from nitrogen, oxygen and sulfur. The heterocyclyl radical may be bonded to the rest of the molecule via a carbon atom or heteroatom. Examples of heterocyclyl include, but are not limited to, azetidinyl, oxetanyl, pyrrolinyl, pyrrolidyl, tetrahydrofuryl, tetrahydrothienyl, piperidyl, piperazinyl, tetrahydropyranyl, morpholinyl or perhydroazepinyl.

As used herein, the term “benzyl” refers to a —CH₂Ph group.

The compounds of Formula (II) have as their “X” substituent a protecting group to protect the amine nitrogen from chemical modification during the synthesis of the compounds of Formula (I) (see synthetic schemes below).

Preferably, in the compounds according to Formula (I) and Formula (II), R¹ and R² are each independently C₁-C₃alkyl (ie, methyl, ethyl, n-propyl, or isopropyl). Even more preferably, R¹ and R² are each independently methyl or ethyl. Most preferably, R¹ and R² are methyl. When R² is C₁-C₃alkoxy, preferably it is methoxy.

Preferably, in the compounds according to Formula (II), X is C₁-C₆alkyl, C₁-C₆alkoxy, hydroxyl, amine, N—C₁-C₆alkylamino, N,N-di-C₁-C₆alkylamino, C₁-C₆alkylcarbonyl, C₁-C₆alkoxycarbonyl, aryl, heteroaryl, heterocyclyl or benzyl, wherein each of C₁-C₆alkyl, C₁-C₆alkoxy, aryl, heteroaryl, heterocyclyl or benzyl may be substituted by 1 to 3 cyano, nitro, halogen, C₁-C₆alkyl, C₁-C₆alkoxy, C₁-C₆haloalkyl, C₂-C₆alkenyl or C₂-C₆alkynyl groups. More preferably, X is C₁-C₆alkyl, C₁-C₆alkylcarbonyl, C₁-C₆alkoxycarbonyl, aryl, heteroaryl, heterocyclyl, or benzyl, or C₁-C₆alkoxy, aryl, heteroaryl, heterocyclyl or benzyl substituted by a cyano, nitro, halogen, C₁-C₆alkyl, C₁-C₆alkoxy, C₁-C₆haloalkyl, C₂-C₆alkenyl or C₂-C₆alkynyl group. Even more preferably, X is C₁-C₆alkoxycarbonyl. Most preferably, X is tert-butoxycarbonyl.

Preferably, the compound of Formula (I) is a compound of Formula (IA-1a) or (IA-1b):

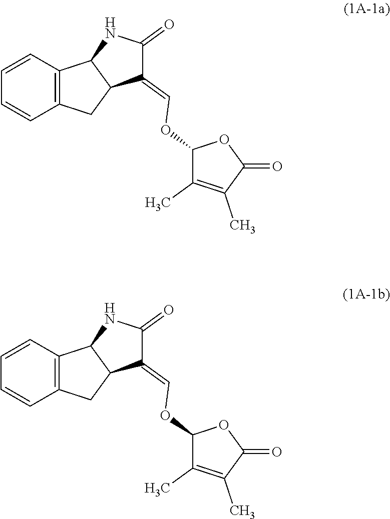 [SMILES: CC1=C(C)[C@H](O/C=C2/C(=O)N[C@@H]3c4ccccc4C[C@H]23)OC1=O ; CC1=C(C)[C@@H](O/C=C2/C(=O)N[C@@H]3c4ccccc4C[C@H]23)OC1=O]

More preferably, the compound of Formula (I) is the compound of Formula (IA-1a).

Table 1 below includes examples A-1 to A-32 of compounds of Formula (I) according to the invention:

TABLE 1

| | |
|---|---|
| | (I) |
| 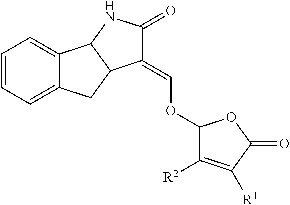 | |

| Compound | R¹ | R² |
|---|---|---|
| A-1 | —CH₃ | —CH₃ |
| A-2 | —CH₃ | —C₂H₅ |
| A-3 | —CH₃ | —CH₂CH₂CH₃ |
| A-4 | —CH₃ | —CH(CH₃)₂ |
| A-5 | —C₂H₅ | —CH₃ |
| A-6 | —C₂H₅ | —C₂H₅ |
| A-7 | —C₂H₅ | —CH₂CH₂CH₃ |
| A-8 | —C₂H₅ | —CH(CH₃)₂ |
| A-9 | —CH₂CH₂CH₃ | —CH₃ |
| A-10 | —CH₂CH₂CH₃ | —C₂H₅ |
| A-11 | —CH₂CH₂CH₃ | —CH₂CH₂CH₃ |
| A-12 | —CH₂CH₂CH₃ | —CH(CH₃)₂ |
| A-13 | —CH(CH₃)₂ | —CH₃ |
| A-14 | —CH(CH₃)₂ | —C₂H₅ |
| A-15 | —CH(CH₃)₂ | —CH₂CH₂CH₃ |
| A-16 | —CH(CH₃)₂ | —CH(CH₃)₂ |
| A-17 | —CH₃ | —OCH₃ |
| A-18 | —C₂H₅ | —OCH₃ |
| A-19 | —CH₂CH₂CH₃ | —OCH₃ |
| A-20 | —CH(CH₃)₂ | —OCH₃ |
| A-21 | —CH₃ | —OC₂H₅ |
| A-22 | —C₂H₅ | —OC₂H₅ |
| A-23 | —CH₂CH₂CH₃ | —OC₂H₅ |
| A-24 | —CH(CH₃)₂ | —OC₂H₅ |
| A-25 | —CH₃ | —OCH₂CH₂CH₃ |
| A-26 | —C₂H₅ | —OCH₂CH₂CH₃ |
| A-27 | —CH₂CH₂CH₃ | —OCH₂CH₂CH₃ |
| A-28 | —CH(CH₃)₂ | —OCH₂CH₂CH₃ |
| A-29 | —CH₃ | —OCH(CH₃)₂ |
| A-30 | —C₂H₅ | —OCH(CH₃)₂ |
| A-31 | —CH₂CH₂CH₃ | —OCH(CH₃)₂ |
| A-32 | —CH(CH₃)₂ | —OCH(CH₃)₂ |

Preferably, the plant growth regulator or seed germination promoting composition according to the invention is a composition that is a seed treatment composition or a seed coating composition. The compositions according to the invention may also further comprise an insecticidal, acaracidal, nematicidal or fungicidal active ingredient.

Preferably, the use of the compound of Formula (I) according to the invention is use in a seed treatment composition, in particular under drought stress conditions or cold stress conditions.

Preferably, the plant propagation material of the invention is a seed. More preferably, a corn (maize) seed.

The compound of Formula (I) according to the invention can be used as a plant growth regulator or seed germination promoter by itself, but is generally formulated into a plant growth regulation or seed germination promotion composition using formulation adjuvants, such as carriers, solvents and surface-active agents (SFAs). The composition can be in the form of concentrates which are diluted prior to use, although ready-to-use compositions can also be utilised. The final dilution is usually made with water, but can be made instead of, or in addition to, water, with, for example, liquid fertilisers, other active ingredients (eg, insecticidal, acaracidal, nematacidal or fungicidal components), micronutrients, biological organisms, oil or solvents.

The compositions generally comprise from 0.1 to 99% by weight, especially from 0.1 to 95% by weight, of a compound of Formula (I) and from 1 to 99.9% by weight of a formulation adjuvant, which preferably includes from 0 to 25% by weight of an SFA.

The compositions can be chosen from a number of formulation types, many of which are known from the Manual on Development and Use of FAO Specifications for Plant Protection Products, 5th Edition, 1999.

These include dustable powders (DP), soluble powders (SP), water soluble granules (SG), water dispersible granules (WG), wettable powders (WP), granules (GR) (slow or fast release), soluble concentrates (SL), oil miscible liquids (OL), ultra low volume liquids (UL), emulsifiable concentrates (EC), dispersible concentrates (DC), emulsions (both oil in water (EW) and water in oil (EO)), micro-emulsions (ME), suspension concentrates (SC), aerosols, capsule suspensions (CS) and seed treatment formulations. The formulation type chosen in any instance will depend upon the particular purpose envisaged and the physical, chemical and biological properties of the compound of Formula (I).

Dustable powders (DP) may be prepared by mixing a compound of Formula (I) with one or more solid diluents (for example natural clays, kaolin, pyrophyllite, bentonite, alumina, montmorillonite, kieselguhr, chalk, diatomaceous earths, calcium phosphates, calcium and magnesium carbonates, sulfur, lime, flours, talc and other organic and inorganic solid carriers) and mechanically grinding the mixture to a fine powder.

Soluble powders (SP) may be prepared by mixing a compound of Formula (I) with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of Formula (I) with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of Formula (I) and one or more powdered solid diluents or carriers, or from pre-formed blank granules by absorbing a compound of Formula (I) (or a solution thereof, in a suitable agent) in a porous granular material (such as pumice, attapulgite clays, fuller's earth, kieselguhr, diatomaceous earths or ground corn cobs) or by adsorbing a compound of Formula (I) (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of Formula (I) in water or an organic solvent, such as a ketone, alcohol or glycol ether. These solutions may contain a surface active agent (for example to improve water dilution or prevent crystallisation in a spray tank).

Emulsifiable concentrates (EC) or oil-in-water emulsions (EW) may be prepared by dissolving a compound of Formula (I) in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment.

Preparation of an EW involves obtaining a compound of Formula (I) either as a liquid (if it is not a liquid at room temperature, it may be melted at a reasonable temperature, typically below 70° C.) or in solution (by dissolving it in an appropriate solvent) and then emulsifying the resultant liquid or solution into water containing one or more SFAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SFAs, to produce spontaneously a thermodynamically stable isotropic liquid formulation. A compound of Formula (I) is present initially in either the water or the solvent/SFA blend. Suitable solvents for use in MEs include those hereinbefore described for use in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of Formula (I). SCs may be prepared by ball or bead milling the solid compound of Formula (I) in a suitable medium, optionally with one or more dispersing agents, to produce a fine particle suspension of the compound. One or more wetting agents may be included in the composition and a suspending agent may be included to reduce the rate at which the particles settle. Alternatively, a compound of Formula (I) may be dry milled and added to water, containing agents hereinbefore described, to produce the desired end product.

Aerosol formulations comprise a compound of Formula (I) and a suitable propellant (for example n-butane). A compound of Formula (I) may also be dissolved or dispersed in a suitable medium (for example water or a water miscible liquid, such as n-propanol) to provide compositions for use in non-pressurised, hand-actuated spray pumps.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of Formula (I) and, optionally, a carrier or diluent therefor. The polymeric shell may be produced by either an interfacial polycondensation reaction or by a coacervation procedure. The compositions may provide for controlled release of the compound of Formula (I) and they may be used for seed treatment. The compound of Formula (I) may also be formulated in a biodegradable polymeric matrix to provide a slow, controlled release of the compound.

The composition may include one or more additives to improve the biological performance of the composition, for example by improving wetting, retention or distribution on surfaces; resistance to rain on treated surfaces; or uptake or mobility of the compound of Formula (I). Such additives include SFAs, spray additives based on oils, for example certain mineral oils or natural plant oils (such as soy bean and rape seed oil), and blends of these with other bio-enhancing adjuvants (ingredients which may aid or modify the action of a compound of Formula (I)).

Wetting agents, dispersing agents and emulsifying agents may be SFAs of the cationic, anionic, amphoteric or non-ionic type.

Suitable SFAs of the cationic type include quaternary ammonium compounds (for example cetyltrimethyl ammonium bromide), imidazolines and amine salts.

Suitable anionic SFAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefine sulphonates, taurates and lignosulphonates.

Suitable SFAs of the amphoteric type include betaines, propionates and glycinates.

Suitable SFAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); and lecithins.

Suitable suspending agents include hydrophilic colloids (such as polysaccharides, polyvinylpyrrolidone or sodium carboxymethylcellulose) and swelling clays (such as bentonite or attapulgite).

In addition, further, other biocidally-active ingredients or compositions may be combined with the compositions of the invention and used in the methods of the invention and applied simultaneously or sequentially with the compositions of the invention. When applied simultaneously, these further active ingredients may be formulated together with the compositions of the invention or mixed in, for example, the spray tank. These further biocidally active ingredients may be fungicides, insecticides, bactericides, acaricides, nematicides and/or other plant growth regulators. Pesticidal agents are referred to herein using their common name are known, for example, from “The Pesticide Manual”, 15th Ed., British Crop Protection Council 2009.

In the methods for regulating the growth of plants in a locus and for promoting the germination of seeds according to the present invention, the application is generally made by spraying the composition, typically by tractor mounted sprayer for large areas, but other methods such as dusting (for powders), drip or drench can also be used. Alternatively, the composition may be applied in furrow or directly to a seed before or at the time of planting. In the method for promoting the germination of seeds according to the present invention, the compound of Formula (I) may be incorporated as a component in a seed treatment composition.

The compound of Formula (I) or composition of the present invention may be applied to a plant, part of the plant, plant organ, plant propagation material or a surrounding area thereof.

In one embodiment, the invention relates to a method of treating a plant propagation material comprising applying to the plant propagation material a composition of the present invention in an amount effective to promote germination and/or regulate plant growth. The invention also relates to a plant propagation material treated with a compound of Formula (I) or a composition of the present invention. Preferably, the plant propagation material is a seed.

The term “plant propagation material” denotes all the generative parts of the plant, such as seeds, which can be used for the multiplication of the latter and vegetative plant materials such as cuttings and tubers. In particular, there may be mentioned the seeds, roots, fruits, tubers, bulbs, and rhizomes.

Methods for applying active ingredients to plant propagation material, especially seeds, are known in the art, and include dressing, coating, pelleting and soaking application methods of the propagation material. The treatment can be applied to the seed at any time between harvest of the seed and sowing of the seed or during the sowing process. The seed may also be primed either before or after the treatment. The compound of Formula (I) may optionally be applied in combination with a controlled release coating or technology so that the compound is released over time.

The composition of the present invention may be applied pre-emergence or post-emergence. Suitably, where the composition is being used to regulate the growth of crop plants, it may be applied pre- or post-emergence, but preferably post-emergence of the crop. Where the composition is used to promote the germination of seeds, it may be applied pre-emergence.

The rates of application of the compound of Formula (I) may vary within wide limits and depend on the nature of the soil, the method of application (pre- or post-emergence; seed dressing; application to the seed furrow; no tillage application, etc.), the crop plant, the prevailing climatic conditions, and other factors governed by the method of application, the time of application and the target crop. For foliar or drench application, the compound of Formula (I) according to the invention is generally applied at a rate of from 1 to 2000 g/ha, especially from 5 to 1000 g/ha. For seed treatment, the rate of application is generally between 0.0005 and 150 g per 100 kg of seed.

Plants in which the composition according to the invention can be used include crops such as cereals (for example wheat, barley, rye, oats); beet (for example sugar beet or fodder beet); fruits (for example pomes, stone fruits or soft fruits, such as apples, pears, plums, peaches, almonds, cherries, strawberries, raspberries or blackberries); leguminous plants (for example beans, lentils, peas or soybeans); oil plants (for example rape, mustard, poppy, olives, sunflowers, coconut, castor oil plants, cocoa beans or groundnuts); cucumber plants (for example marrows, cucumbers or melons); fibre plants (for example cotton, flax, hemp or jute); citrus fruit (for example oranges, lemons, grapefruit or mandarins); vegetables (for example spinach, lettuce, asparagus, cabbages, carrots, onions, tomatoes, potatoes, cucurbits or paprika); lauraceae (for example avocados, cinnamon or camphor); maize; rice; tobacco; nuts; coffee; sugar cane; tea; vines; hops; durian; bananas; natural rubber plants; turf or ornamentals (for example flowers, shrubs, broad-leaved trees or evergreens such as conifers). This list does not represent any limitation.

The invention may also be used to regulate the growth, or promote the germination of seeds of non-crop plants, for example to facilitate weed control by synchronizing germination.

Crops are to be understood as also including those crops which have been modified by conventional methods of breeding or by genetic engineering. For example, the invention may be used in conjunction with crops that have been rendered tolerant to herbicides or classes of herbicides (e.g. ALS-, GS-, EPSPS-, PPO-, ACCase- and HPPD-inhibitors). An example of a crop that has been rendered tolerant to imidazolinones, e.g., imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®. Methods of rendering crop plants tolerant to HPPD-inhibitors are known; for example the crop plant is transgenic in respect of a polynucleotide comprising a DNA sequence which encodes an HPPD-inhibitor resistant HPPD enzyme derived from a bacterium, more particularly from Pseudomonas fluorescens or Shewanella colwelliana, or from a plant, more particularly, derived from a monocot plant or, yet more particularly, from a barley, maize, wheat, rice, Brachiaria, Chenchrus, Lolium, Festuca, Setaria, Eleusine, Sorghum or Avena species.

Crops are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). The Bt toxin is a protein that is formed naturally by Bacillus thuringiensis soil bacteria. Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crops are also to be understood to include those which are obtained by conventional methods of breeding or genetic engineering and contain so-called output traits (e.g., improved storage stability, higher nutritional value and improved flavour).

The compound of the invention may be prepared by any of the general methods disclosed in WO 2012/080115.

### EXAMPLES

The Examples which follow serve to illustrate the invention.

### Compound Synthesis and Characterisation

The following abbreviations are used throughout this section: s=singlet; bs=broad singlet; d=doublet; dd=double doublet; dt=double triplet; bd=broad doublet; t=triplet; dt=double triplet; bt=broad triplet; tt=triple triplet; q=quartet; m=multiplet; Me=methyl; Et=ethyl; Pr=propyl; Bu=butyl; DME=1,2-dimethoxyethane; M.p.=melting point; RT=retention time, MH⁺=molecular cation (i.e. measured molecular weight).

The following HPLC-MS method was used for the analysis of the compounds: Spectra were recorded on a ZQ Mass Spectrometer from Waters (Single quadrupole mass spectrometer) equipped with an electrospray source (Polarity: positive or negative ions, Capillary: 3.00 kV, Cone: 30.00 V, Extractor: 2.00 V, Source Temperature: 100° C., Desolvation Temperature: 250° C., Cone Gas Flow: 50 L/Hr, Desolvation Gas Flow: 400 L/Hr, Mass range: 100 to 900 Da and an Acquity UPLC from Waters (Solvent degasser, binary pump, heated column compartment and diode-array detector. Column: Waters UPLC HSS T3, 1.8 μm, 30×2.1 mm, Temp: 60° C., flow rate 0.85 mL/min; DAD Wavelength range (nm): 210 to 500) Solvent Gradient: A=H₂O+ 5% MeOH+ 0.05% HCOOH, B=Acetonitrile+ 0.05% HCOOH) gradient: 0 min 10% B; 0-1.2 min 100% B; 1.2-1.50 min 100% B.

Compounds of the invention were prepared in accordance with Preparation Examples 1 to 3.

### Preparation Example 1

### tert-butyl (3E,3aR,8bS)-3-[[(2R)-3,4-dimethyl-5-oxo-2H-furan-2-yl]oxymethylene]-2-oxo-4,8b-dihydro-3aH-indeno[1,2-b]pyrrole-1-carboxylate (compound of Formula (IIA-1a)); and

### tert-butyl (3E,3aR,8bS)-3-[[(2S)-3,4-dimethyl-5-oxo-2H-furan-2-yl]oxymethylene]-2-oxo-4,8b-dihydro-3aH-indeno[1,2-b]pyrrole-1-carboxylate (compound of Formula (IIA-1b))

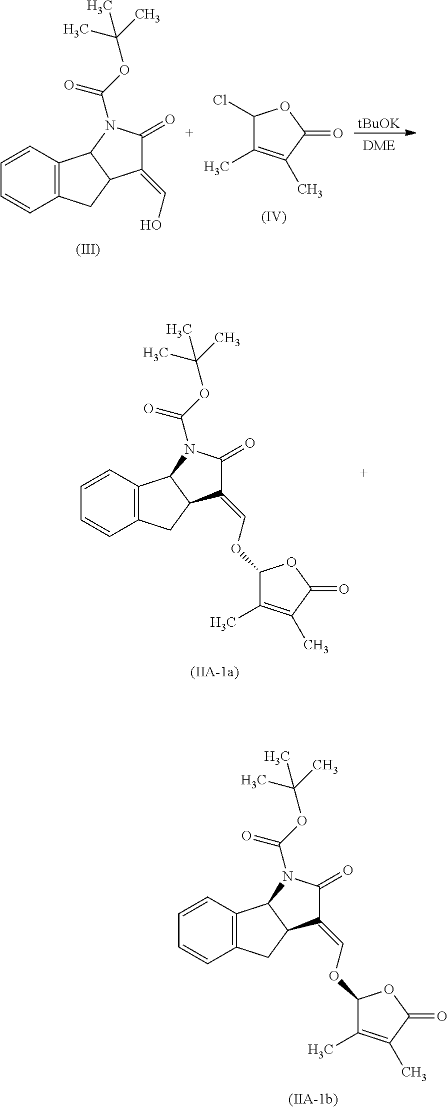

To a cooled solution of known compound (III) (refer WO 2012/080115) (5.5 g) in 1,2-dimethoxyethane (DME, 150 mL) under an argon atmosphere, was added tBuOK (2.5 g, 1.20 eq). The reaction mixture was then stirred at 0° C. for 5 min prior to the addition of a solution of known compound (IV) (refer WO 2012/056113) (2.9 g, 1.1 eq) in DME (5 mL). The resulting reaction mixture was stirred for 15 min at 0° C. then slowly warmed to room temperature. After 16 h at room temperature, the reaction mixture was diluted with water and ethyl acetate. The phases were separated, the aqueous layer extracted with ethyl acetate and the combined organic layers were washed with brine, dried over Na₂SO₄ and concentrated under vacuum. The crude product was purified by chromatography on silica gel (SiO₂) affording (IIA-1a) and (IIA-1b) as two separate diastereoisomers in 69% yield (combined yield).

(IIA-1a) LCMS: RT 1.09 min; ES+ 412 (M+H⁺); ¹H NMR (400 MHz, CDCl₃) δ 1.62 (s, 9H), 1.91 (m, 3H), 2.05 (m, 3H), 3.17 (dd, 1H), 3.36 (dd, 1H), 3.77 (m, 1H), 5.71 (d, 1H), 5.95 (bs, 1H), 7.17-7.31 (m, 3H), 7.39 (d, 1H), 7.67 (d, 1H).

(IIA-1b) LCMS: RT 1.08 min; ES⁺ 412 (M+H⁺); ¹H NMR (400 MHz, CDCl₃) 1.61 (s, 9H), 1.91 (m, 3H), 2.02 (m, 3H), 3.16 (d, 1H), 3.33 (dd, 1H), 3.76 (m 1H), 5.71 (d, 1H), 5.94 (bs, 1H), 7.20 (bt, 2H), 7.24-7.29 (m, 1H), 7.36 (d, 1H), 7.65 (bd, 1H).

### Preparation Example 2

### (3E,3aR,8bS)-3-[[(2R)-3,4-dimethyl-5-oxo-2H-furan-2-yl]oxymethylene]-1,3a,4,8b-tetrahydroindeno[1,2-b]pyrrol-2-one (compound of Formula (IA-1a))

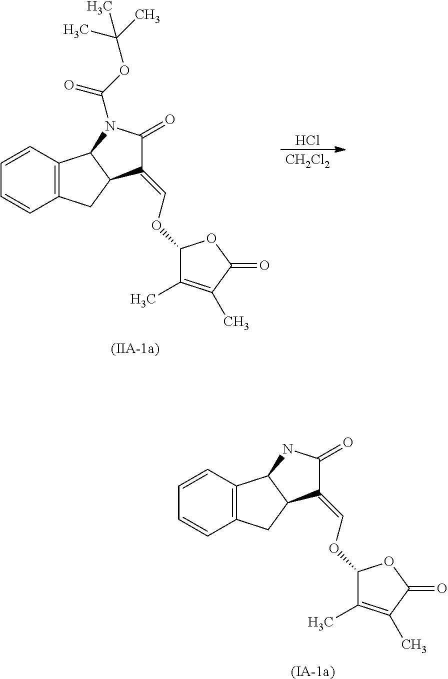

Compound (IIA-1a) was dissolved in CH₂Cl₂ and HCl (2M in Et₂O) was added dropwise. The resulting reaction mixture was stirred for 15 min at room temperature and then poured into an aqueous NaHCO₃ solution. The organic phase was extracted with CH₂Cl₂ and the combined organic layers were washed with brine, dried over Na₂SO₄ and concentrated under vacuum affording compound (IA-1a) in 98% yield.

LCMS: RT 0.86 min; ES⁺ 312 (M+H⁺); ¹H NMR (400 MHz, CDCl₃) δ 1.91 (m, 3H), 2.05 (m, 3H), 3.10 (dd, 1H), 3.48 (dd, 1H), 3.94 (m, 1H), 5.12 (bd, 1H), 5.94 (bs, 1H), 5.98 (bs, 1H), 7.20-7.32 (m, 4H).

### Preparation Example 3

### (3E,3aR,8bS)-3-[[(2S)-3,4-dimethyl-5-oxo-2H-furan-2-yl]oxymethylene]-1,3a,4,8b-tetrahydroindeno[1,2-b]pyrrol-2-one (compound of Formula (IA-1b))

Compound (IA-1b) was prepared following the same procedure as compound (IA-1a) as described above in Preparation Example 2.

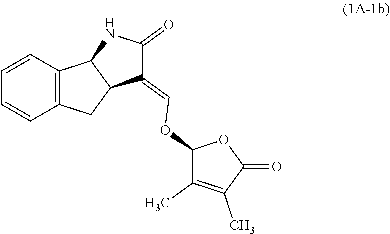 [SMILES: CC1=C(C)[C@@H](O/C=C2/C(=O)N[C@@H]3c4ccccc4C[C@H]23)OC1=O]

LCMS: RT 0.85 min; ES⁺ 312 (M+H⁺); ¹H NMR (400 MHz, CDCl₃) δ 1.91 (m, 3H), 2.05 (m, 3H), 3.10 (dd, 1H), 3.48 (dd, 1H), 3.94 (m, 1H), 5.12 (bd, 1H), 5.94 (bs, 1H), 5.98 (bs, 1H), 7.20-7.32 (m, 4H).

### Preparation Example 4

### (3E,3aR,8bS)-3-[[(2R)-3-methoxy-4-methyl-5-oxo-2H-furan-2-yl]oxymethylene]-1,3a,4,8b-tetrahydroindeno[1,2-b]pyrrol-2-one (compound of Formula (IA-17a)); and

### (3E,3aR,8bS)-3-[[(2S)-3-methoxy-4-methyl-5-oxo-2H-furan-2-yl]oxymethylene]-1,3a,4,8b-tetrahydroindeno[1,2-b]pyrrol-2-one (compound of Formula (IA-17b))

Compounds (IA-17a) and IA-17b) were prepared following the same procedure as compound (IA-1a and IA-1b) as described above in Preparation Example 1-3 from compound (III) and known 2-hydroxy-3-methoxy-4-methyl-2H-furan-5-one (WO 2013/171092).

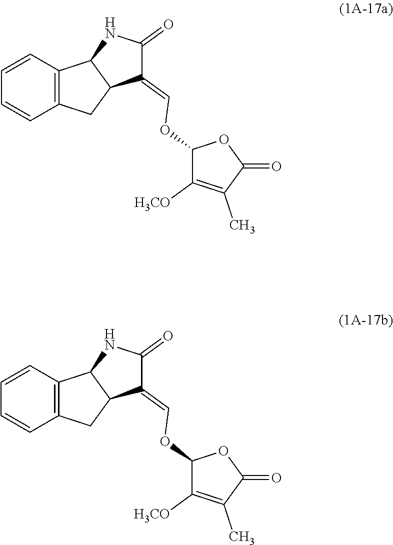 [SMILES: COC1=C(C)C(=O)O[C@H]1O/C=C1/C(=O)N[C@@H]2c3ccccc3C[C@H]12 ; COC1=C(C)C(=O)O[C@@H]1O/C=C1/C(=O)N[C@@H]2c3ccccc3C[C@H]12]

(IA-17a) LCMS: RT 0.83 min; ES+ 328 (M+H⁺); ¹H NMR (400 MHz, CDCl₃) δ 1.98 (s, 3H), 3.09 (dd, 1H), 3.49 (dd, 1H), 3.95 (m, 1H), 4.13 (s, 3H), 5.12 (bd, 1H), 5.93 (s, 1H), 6.21 (bs, 1H), 7.19-7.33 (m, 4H).

(IA-17b) LCMS: RT 0.83 min; ES⁺ 328 (M+H⁺); ¹H NMR (400 MHz, CDCl₃) δ 1.98 (s, 3H), 3.11 (dd, 1H), 3.47 (dd, 1H), 3.95 (m, 1H), 4.12 (s, 3H), 5.11 (d, 1H), 5.92 (s, 1H), 6.24 (bs, 1H), 7.19-7.30 (m, 4H).

### Stability Studies

Comparative soil stability and hydrolytic stability studies were conducted on compounds according to the invention (Compounds (IA-1a) and (IA-1b)) and structurally-related compounds known from the prior art (Compounds P1 and P2 disclosed in WO 2012/080115—see below).

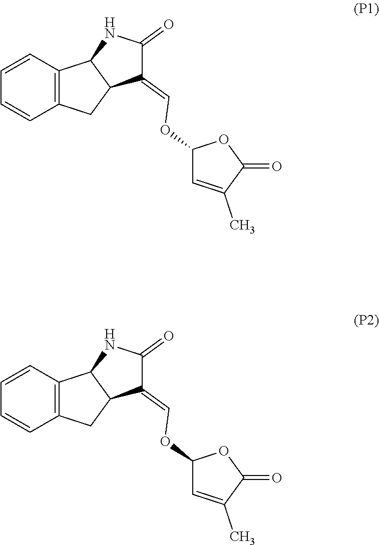 [SMILES: CC1=C[C@H](O/C=C2/C(=O)N[C@@H]3c4ccccc4C[C@H]23)OC1=O ; CC1=C[C@@H](O/C=C2/C(=O)N[C@@H]3c4ccccc4C[C@H]23)OC1=O]

### Example 4—Hydrolytic Stability Assay

The objective of the hydrolytic stability assays is to determine the chemical stability of individual test compounds according to the invention in a controlled and reproducible environment, allowing a comparison of compound in vitro stability under aqueous conditions at pH 7 and 9.

### Sample Preparations

### Standard Solutions/Treatment Solution

Prior to conducting the individual hydrolytic stability assays, stock solutions containing 1000 ppm of each test compound (ie, compounds (IA-1a), (IA-1b), (IA-17a), (IA-17b), P1 and P2) were prepared in methanol.

The reagents used in the assays were prepared as follows. A 20 mM buffer solution was prepared from a stock solution of 20 mM mixed acetate, borate and phosphate buffer and the pH adjusted to 7 or 9 as required.

Test solutions were prepared in LC vials for each test compound in the following manner:

Mobile Phase Control: Mobile phase (1 mL)+compound stock solution (2 to 40 μL).

Hydrolytic Stability: Buffer (1 mL)+compound stock solution (2 to 40 μL).

The mobile phase and buffer were initially dispensed into separate glass LC vials, placed into an autosampler complete with thermostat set at 40° C. and allowed to equilibrate for 30 minutes prior to starting the individual assays.

Reactions were initiated by addition of the compound solution and monitored through a series of repeat injections made directly from the vial into the HPLC system at regular time intervals. Initial and subsequent measurements of peak area attributable to the test compound were used to fit exponential half-lives and calculate first-order rate constants. Definitive half-lives could not be determined for test compounds (IA-1a and IA-17a) and (IA-1b and IA-17b) at pH 7, as insufficient loss was observed under the experimental conditions employed. Consequently, the percentage of compound remaining was recorded at the last assessment time.

Stability data (t_{1/2}), ie, the time in hours for half of the test compound to be hydrolysed, are provided in Table 3 below.

### Example 5—Soil Stability Assay

It is highly desirable that agrochemicals applied to soil in order to deliver a beneficial biological effect can exist in the soil for a prolonged period of time with minimal degradation. However, a biologically active agrochemical compound may undergo chemical transformation in soil, leading to decreased levels of activity and a decrease in a desired biological effect. Simple laboratory degradation studies can be used to evaluate the disappearance due to biotic and abiotic processes of a compound in soil. The time taken for a compound to degrade in soil allows the estimation of their half-life (t_{1/2}), which corresponds to the time in which 50% of the compound under evaluation is degraded in soil. This can be a useful parameter to evaluate the stability of a compound in soil, with the longer the half-life, the more stable the compound.

### Sample Preparations

### Standard Solutions/Treatment Solution

Stock standard solutions were prepared by dissolving 1 mg of each test compound (ie, compounds (IA-1a and IA-1b), (IA-17a and IA-17b), P1 and P2) in acetonitrile. The stock standard solutions were stored at 6° C. Working standard solutions were then obtained by a series of dilutions of the stock standard solutions. A treatment solution of 100 μg/mL concentration for each test compound was prepared in methanol.

### Soil Preparation

Soil samples used for this soil stability assay were collected at the Syngenta Research Centre location in Stein (Switzerland). The soil was classified as clay loam. Certain physical properties of the soil are described in Table 2.

TABLE 2
Physical properties of Stein soil.

| | | | | | | Cation | Water Hold | Water Hold |
|---|---|---|---|---|---|---|---|---|
| | | | | | Organic | Exchange | Capacity at | Capacity at |
| Water | CaCl₂ | Sand | Silt | Clay | Matter | Capacity | 0.33 bar | 15 bar |
| pH | pH | % | % | % | % | M eq/100 g | % | % |
| 7.6 | 7.3 | 37 | 35 | 28 | 3.8 | 19.4 | 25.8 | 14.8 |

2 mm sieved Stein soil was mixed with sand at ratio 1:1 prior to starting the laboratory soil degradation experiment. 10 g of the sand-soil mix (air-dried basis) was weighed into 50 ml Corning® polypropylene centrifuge tubes and soil moisture was adjusted at 45% of the field capacity.

### Chemical Application and Incubation Conditions

Chemical application was performed by applying 30 μl of a 100 μg/mL solution of each test compound to 10 g soil vessel corresponding to a final concentration of 0.3 μg test compound per gram of soil. Three replicates were considered for each test compound. Treated tubes were incubated in the dark at 20° C.±0.5. For the estimation of half-life, different sampling times were considered.

Based on initial studies, a fast degradation was expected for compounds P1, P2 and IA-17 therefore the following short sampling times of 0, 3, 6, 9 and 24 hours after application were considered. Conversely for compounds IA-1, longer sampling times of 0, 6, 24, 48, 72, 168 and 240 hours after application were used. At each sampling time, samples were removed and stored at −80° C. until analysis. The half-lives were calculated by an exponential regression analysis plotting the percentage of recovered compound in soil against the time.

### Chemical Extraction and Analysis

Compounds P1, P2, (IA-1) and (IA-17) were extracted from soil by using 30 mL of Acetonitrile (CHROMASOLV® gradient grade, for HPLC, >99.9%, SIGMA-ALDRICH). The mixture was shaken for 3 hours at room temperature by using a vertical rotary shaker. After centrifugation at 3000 rpm for 5 minutes, an aliquot of the supernatant was collected and analyzed via UPLC-MS (Waters Acquity UPLC-MS PDA-Detection: 254 nm- and SQD-Zspray ESI, APCI, ESCi®-; Waters Acquity UPLC Column HSS T3 2.1×30 mm-1.8 μm; Gradient mobile phase with Solvent A: Water/MeOH (9:1)+ 0.1% Formic Acid and Solvent B: Acetonitrile+ 0.1% Formic Acid; flow of 0.75 ml/min).

Compounds P1 and P2, are monomethyl analogues of the compounds of Formula (IA-1a and IA-1b) according to the invention. As with the compounds according to the invention, compounds P1 and P2 show seed germination promotion properties. However, as Table 3 shows, in comparison to compounds P1 and P2, the compounds of Formula (IA-1a and IA-1b) and (IA-17a and IA-17b) of the present invention show surprising and unexpectedly superior levels of both soil stability and hydrolytic stability.

TABLE 3
Stability data of compounds (IA-1a and IA-1b) and (IA-17a and IA-17b)
(disubstituted butenolide) versus compounds P1 and P2 of the prior art (monomethyl
butenolide)

| | | | Hydrolytic Stability | | |
|---|---|---|---|---|---|
| | | Literature | (t_{1/2}, hours) | | Soil Stability |
| Compound | | reference | pH 7 | pH 9 | (t_{1/2}, hours) |
| 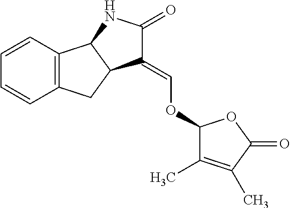 [SMILES: CC1=C(C)[C@@H](O/C=C2/C(=O)N[C@@H]3c4ccccc4C[C@H]23)OC1=O] | (IA-1b) | — | stable^{a} | 2.1 | 53 |
| 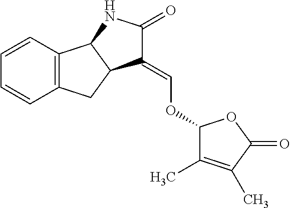 [SMILES: CC1=C(C)[C@H](O/C=C2/C(=O)N[C@@H]3c4ccccc4C[C@H]23)OC1=O] | (IA-1a) | — | stable^{b} | 2.4 | 87 |
| 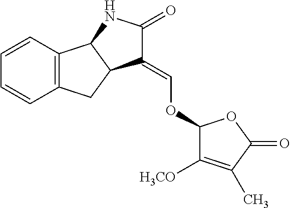 [SMILES: COC1=C(C)C(=O)O[C@@H]1O/C=C1/C(=O)N[C@@H]2c3ccccc3C[C@H]12] | (IA-17b) | — | stable^{c} | 19.0 | 312 |
| 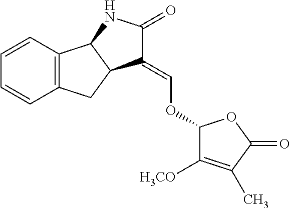 [SMILES: COC1=C(C)C(=O)O[C@H]1O/C=C1/C(=O)N[C@@H]2c3ccccc3C[C@H]12] | (IA-17a) | — | stable^{d} | 16.6 | 340 |
| 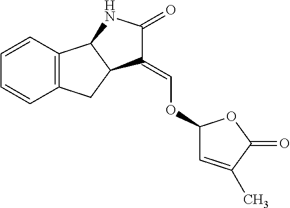 [SMILES: CC1=C[C@@H](O/C=C2/C(=O)N[C@@H]3c4ccccc4C[C@H]23)OC1=O] | P2 | WO 2012/080115 | 79.5^{e} | 0.3 | 4.2 |
| 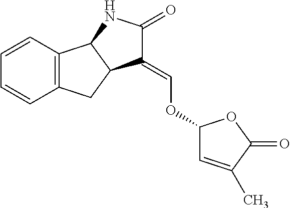 [SMILES: CC1=C[C@H](O/C=C2/C(=O)N[C@@H]3c4ccccc4C[C@H]23)OC1=O] | P1 | WO 2012/080115 | 25.6^{f} | 0.4 | 4 |

^{a}90.9% remaining at 21.1 hours
^{b}91.8% remaining at 17.9 hours
^{c}99.0% remaining at 16.8 hours
^{d}99.1% remaining at 17.3 hours
^{e}86.9% remaining at 15.8 hours
^{f}55.4% remaining at 21.5 hours

As can be seen from Table 3, the compounds of the present invention show superior hydrolytic stability to the prior art compounds at the biologically-relevant pH levels of pH 7 and 9. Likewise, compound of the present invention shows superior soil stability compared to the prior art compounds.

### Biological Examples

### Corn Seed Germination

Corn seed germination studies were undertaken on the compounds of the present invention. In particular, the effect of the compounds of Formulae (IA-1a) and (IA-1b) on the germination of NK Falkone corn seeds (Syngenta) under cold stress was evaluated as follows.

NK Falkone corn seeds were sorted by size using 2 sieves, one excluding very big seeds and the other with round holes of 8 to 9 mm diameter. The seeds retained by the latter sieve were used for the germination test.

The corn seeds were placed in 24 well plates (each plate is considered as one experimental unit or replicate). Germination is initiated by the addition of 250 μl of distilled water containing 0.5% DMSO per well as a means for compound solubilization. 8 replicates (ie, 8 plates) were used for each treatment characterization. Plates were sealed using seal foil (Polyolefin Art. Nr. 900320) from HJ-BIOANALYTIK. All plates were placed horizontally on trolleys in a climatic chamber at 15° C. or 23° C. in complete darkness. The experiment was laid out in a completely randomized design in a climatic chamber with 75% relative humidity. Foils were pierced, one hole per well using a syringe after 72 hours for experiments performed at 15° C. and after 24 hours for experiments performed at 23° C.

Germination was followed over time by taking photographs at different time points. Image analysis is done automatically with a macro developed using the Image J software. A dynamic analysis of germination is carried out by fitting a logistic curve to the relationship between % germination and time for each plate (T₅₀ parameter).

T₅₀ is the time needed for half the seed population to be germinated. A negative value of T₅₀ represents a faster germination rate. The mean of T₅₀ parameters is calculated for the 8 replicates and the kinetic parameter is determined for each germination curve. Data in bold indicate germination enhancing statistically significant differences between treated seeds and control (empty vehicle treated) T₅₀ values (P<0.05) as outlined in Table 4.

TABLE 4
Effect of strigolactam analogue compounds (IA-1a and
IA-1b) and (IA-17a and IA-17b) on germination of corn
seeds under cold stress conditions (15° C.) at 250 μM

| Compound | Rate (μM)^{a} | T₅₀ (% vs control)^{b} |
|---|---|---|
| (IA-1a) | 250 | −6.10^{c} |
| (IA-1b) | 250 | −7.60 |
| (IA-17a) | 250 | −6.50 |
| (IA-17b) | 250 | −5.10 |
| P1 | 250 | −4.20 |
| P2 | 250 | −2.30 |

^{a}Concentration of test compound in 250 μl distilled water containing 0.5% DMSO
^{b}Control = 250 μl distilled water containing 0.5% DMSO; T₅₀ = 110 hours.
^{c}Data in bold are statistically validated

1. A compound of Formula (I): 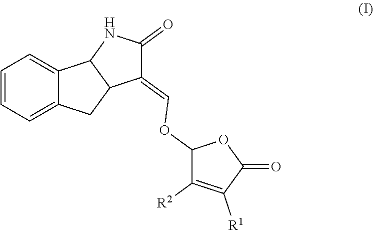
wherein
R¹ is C₁-C₃alkyl; and
R² is C₁-C₃alkyl or C₁-C₃alkoxy;
or a salt or N-oxide thereof.

2. The compound according to claim 1, wherein R¹ and R² are C₁-C₃alkyl.

3. The compound according to claim 1, as defined by Formula (IA-1):

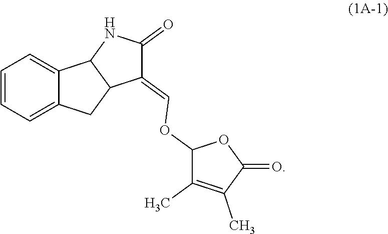 [SMILES: CC1=C(C)C(O/C=C2/C(=O)NC3c4ccccc4CC23)OC1=O]

4. The compound according to claim 1, as defined by Formula (IA-1a) or (IA-1b):

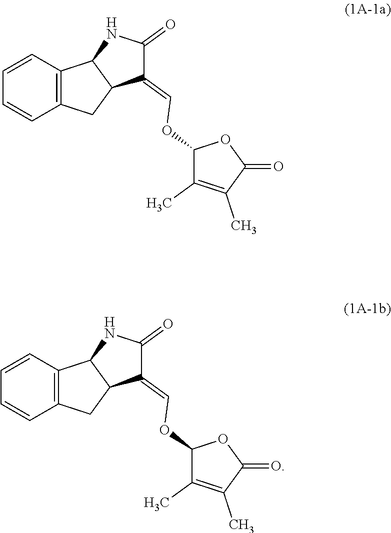 [SMILES: CC1=C(C)[C@H](O/C=C2/C(=O)N[C@@H]3c4ccccc4C[C@H]23)OC1=O ; CC1=C(C)[C@@H](O/C=C2/C(=O)N[C@@H]3c4ccccc4C[C@H]23)OC1=O]

5. A plant growth regulator composition or seed germination promoting composition, comprising the compound according to claim 1, and optionally, an agriculturally acceptable formulation adjuvant.

6. The composition according to claim 5, further comprising an insecticidal, acaracidal, nematicidal or fungicidal active ingredient.

7. The composition according to claim 5, wherein the composition is a seed treatment or a seed coating composition.

8. A method for regulating the growth of plants at a locus, wherein the method comprises applying to the locus a plant growth regulating amount of a composition according claim 5.

9. A method for promoting the germination of seeds comprising applying to the seeds, or a locus containing seeds, a seed germination promoting amount of a composition according claim 5.

10. A method for controlling weeds comprising applying to a locus containing weed seeds a seed germination promoting amount of a composition according claim 5, allowing the seeds to germinate, and then applying to the locus a post-emergence herbicide.

11. (canceled)

12. A method of treating a plant propagation material comprising applying to the plant propagation material a composition according claim 5 in an amount effective to promote germination or regulate plant growth.

13. A plant propagation material treated with a compound of Formula (I) according to claim 1.

14. A compound of Formula (II): 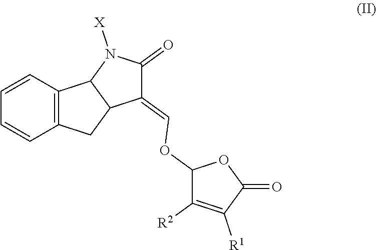
wherein
X is a protecting group;
R¹ is C₁-C₃alkyl; and
R² is C₁-C₃alkyl or C₁-C₃alkoxy.

15. The compound according to claim 14 wherein X is tert-butoxycarbonyl.
